SEQUENCE LISTING (2 sequences)

SEQ ID NO: 1 - DNA, 24 bp, Homo sapiens
tcacaccggaggttacttccctcg

SEQ ID NO: 2 - DNA, 18 bp, Homo sapiens
tccgacgacagcgcgact

(19) United States
(12) Patent Application Publication
(10) Pub. No.: US 2010/0196896 A1
(43) Pub. Date: Aug. 5, 2010

(54) METHOD AND A KIT FOR IDENTIFYING A HUMAN WHO HAS THE PREDISPOSITION FOR INCREASED CONSUMPTION OF CARBOHYDRATES AND METHOD FOR MANAGING THE NAMED HUMAN'S DIETARY INTAKE OF NUTRITIONAL ENERGY

(76) Inventors: Jaanus Harro, Tartu (EE); Jüri Parik, Tartu (EE); Jarek Mäestu, Tartu (EE)

Correspondence Address: REED SMITH LLP, P.O. BOX 488, PITTSBURGH, PA, 15230-0488, US

(21) Appl. No.: 12/597,809

(22) PCT Filed: Apr. 25, 2008
(86) PCT No.: PCT/EE08/00009

§ 371 Date: Feb. 25, 2010

(30) Foreign Application Priority Data
Apr. 27, 2007 (EE) P200700019

(51) Int. Cl. C12Q 1/68

(52) U.S. Cl. 435/6

(57) ABSTRACT

A method and a kit for determining the human with genetic predisposition to increased consumption of carbohydrates and to develop metabolic, psychiatric or neurological disease or disorder, or obesity. The invention pertains to the detection of a human's ADRA2A genotype, and if there is detected the homozygous nucleotide G at the position C-1291 of the ADRA2A gene, then the patient possesses a predisposition to increased consumption of carbohydrates and to develop metabolic, psychiatric or neurological diseases or disorders or obesity, and there is a need to decrease intake of carbohydrates.

### TECHNICAL FIELD OF THE INVENTION

This invention is in the field of molecular biology, more particularly, to nucleic acid sequences a human alpha 2A adrenergic receptor gene (ADRA2A) and the determination of metabolic, psychiatric or neurological disease or disorder, or obesity, and to management of diet to avoid the risk of developing the mentioned diseases or disorders.

### BACKGROUND OF THE INVENTION

Alpha 2A adrenergic receptors are plasma membrane receptors which are located in the peripheral and central nervous systems throughout the human body. Adrenergic receptors are targets for epinephrine and norepinephrine and thus represent a critical component in the sympathetic nervous system for the maintenance of homeostasis and responses to disease. Inhibition of neurotransmitter release in the brain is the basis for the central hypotensive, sedative, anesthetic-sparing, and analgesic responses of alpha-2A adrenoreceptor agonists (Makaritsis et al. Hypertension, 1999, vol 3: 14-17). Adrenergic receptors as widely expressed in the central and peripheral nervous system have been shown to participate in a broad spectrum of physiological functions that include, for example, regulation of blood pressure, inhibition of neurotransmitter release, sedation, analgesia, regulation of insulin release, lipolysis and several behavioral and cognitive functions.

Indeed, alpha-2A adrenoreceptor agonists such as clonidine and guanabenz are potent antihypertensive agents which act via central presynaptic alpha-2A adrenoreceptors (Freeman et al. American journal of Hypertension, 1995, vol 8: 863-869). The blood pressure and other responses to alpha-2A adrenoreceptors agonists and antagonists though, are subject to interindividual variation in the human population (Small et al. Journal of Biological Chemistry, 2001, vol. 276: 4917-4922). Such variation, of course, can be due to genetic variation in the structure of the receptor itself, its cognate G-proteins, the effectors, or downstream intracellular targets. The alpha-2A receptors have been localized in brain, heart, lung, skeletal muscle, blood vessels, kidney, pancreas, prostate, jejunum, ileum, spleen, adrenal gland and spinal cord (Eason et al. Molecular Pharmacology 44, 70-75 (1993).

ADRA2A is published, for example, in U.S. Pat. No. 7,041,810 which discloses polymorphisms in nucleic acids encoding the alpha-2B, alpha-2A, and alpha-2C adrenergic receptor and expressed alpha-2B, alpha-2A and alpha-2C adrenergic receptor molecule. The invention also pertains to methods and molecules for detecting such polymorphisms and methods in the diagnosis, prognosis, and treatment of diseases such as cardiovascular and central nervous system diseases.

International patent application WO2005095984 (Bayer Healthcare AG) describes diagnostics and therapeutics for diseases associated with G protein-coupled alpha 2A-adrenoceptor. However, this application will cover neither the methods to determine the risk for certain disease or the disease itself nor the genotype effect on human's dietary predisposion of consuming sweet carbohydrates.

The nucleotide sequence of ADRA2A is accessible in public databases by the accession number NM000681.

Alpha-2A adrenoreceptors are the principal presynaptic inhibitory autoreceptors of central and peripheral sympathetic nerves and inhibit neurotransmitter release in the brain and cardiac sympathetic nerves. Of particular interest are physiologic and genetic studies which suggest that altered alpha-2A adrenoreceptor function can predispose individuals to different metabolic, psychiatric or neurologic diseases or disorders.

Several physiologic functions of the alpha-2A adrenoreceptors are known in the art. For example, alpha-2A adrenoreceptors act to inhibit insulin secretion by pancreatic beta-cells, contract vascular smooth muscle, inhibit lipolysis in adipocytes, modulate water and electrolyte flux in renal cells, and aggregate platelets (Jewell-Motz, Biochemistry, 1995, vol. 34: 11946-11953). ADRA2A influences also norepinephrine concentrations, blood pressure and heart rate and direct increased expression of ADRA2A may cause alterations in insulin regulatory system and glucose concentration (Devedijan et al. Diabetologia, 2000, vol. 43: 899-906).

### Alpha-2 Adrenergic Receptor Polymorphisms

Alpha-2A adrenoreceptors are widely expressed and participate in a wide spectrum of different physiologic functions that include metabolic, cardiac, vascular, and central and peripheral nervous systems, via pre-synaptic and post-synaptic mechanisms. At peripheral sites, alpha-2A adrenoreceptors act to inhibit insulin secretion by pancreatic beta-cells, contract vascular smooth muscle, inhibit lipolysis in adipocytes, modulate water and electrolyte flux in renal cells, and aggregate platelets.

There may be synonymous or nonsynonymous polymorphisms. Synonymous polymorphisms when present in the coding region typically do not result in an amino acid change. Nonsynonymous polymorphism when present in the coding region alters one or more codons resulting in an amino acid replacement in the amino acid chain. Such mutations and polymorphisms may be either heterozygous or homozygous within an individual. Homozygous individuals have identical alleles at one or more corresponding loci on homologous chromosomes. While heterozygous individuals have two different alleles at one or more corresponding loci on homologous chromosomes. A polymorphism is thus said to be “allelic,” in that, due to the existence of the polymorphism, some members of a species carry a gene with one sequence (e.g., the original or wild-type “allele”), whereas other members may have an altered sequence (e.g., the variant or, mutant “allele”). In the simplest case, only one mutated variant of the sequence may exist, and the polymorphism is said to be diallelic. For example, if the two alleles at a locus are indistinguishable in their effects on the organism, then the individual is said to be homozygous at the locus under consideration. An allele may be referred to by the nucleotide(s) that comprise the mutation (Small et al. U.S. Pat. No. 7,041,810). It is known to those skilled in the art that some other polymorphisms may be in linkage disequilibrium with known polymorphisms behind a particular feature, and therefore the former ones can be used as markers for the feature as well.

A polymorphic site at the position −1291 of the promoter region of the ADRA2A gene was discovered by Lario et al. (Clinical Genetics, 1997, vol. 51. 129-130), where the C allele is substituted with a G allele, may cause more marked increase in body weight with the subjects carrying GG genotype compared to CC or CG genotype when treated with antipsychotic drug clozapine (Wang et al. Journal of Neural Transmission, 2005, vol. 112: 1463-1468). C-1291G polymorphism in the promoter region of the ADRA2A gene alters the regulation of glucose homeostasis, causes higher blood triglyceride levels, and lowers diastolic blood pressure in humans (Rosmond et al. Journal of Internal Medicine 2002; 251: 252-257).

One of the determinants of the excess body weight gain is the increased food intake of young children (Salbe et al. American Journal of Clinical Nutrition, 2003, vol. 78: 193-194). Study of Herbeth et al. (American Journal of Clinical Nutrition, 2005, vol. 82: 467-470) indicated that gene polymorphism could be one of the risk factors of increased food intake.

Alpha 2A adrenergic component is stronger in the abdominal subcutaneous fat depot in obese men compared to lean men, with no difference in femoral depot. In obese women, the alpha 2A adrenergic component is more important in the subcutaneous abdominal and femoral depots (Mauriége et al. Journal of Lipid researh, 1991, vol. 32: 1625-1633, American Journal of Physiology, 1995, vol. 269: 341-350). The effect of G-1291 genotype has been shown to be marginal on fat accumulation differences between C or G allele carriers in Caucasians (Garenc et al. Molecular Medicine, 2002, vol. 8: 88-94). While in black people G allele was associated in 27% increase in trunk to extremity skinfold ratio. In black women G allele presence was associated with 14% decrease in trunk to extremity skinfold ratio and 27% decrease of abdominal visceral fat area. The not observed difference in white people is probably due to the smaller G allele frequency. Thus, polymorphisms of the alpha-2A adrenoreceptors may act as risk factors for disease, act to modify a given disease, or alter the therapeutic response to agonists or antagonists.

Inventions, known to those skilled in the art so far, give no knowledge of the effect of ADRA2A genotype on human's dietary predisposion for consuming sweet carbohydrates. This predisposion may lead to weight gain, metabolic diseases, obesity or psychiatric or neurological diseases or disorders.

### DISCLOSURE OF THE INVENTION

The present invention provides a method and a kit for identifying a human having a predisposition to develop metabolic, psychiatrical or neurological diseases or disorders, comprising detecting a human's ADRA2A genotype and assessing a human's predisposition for increased consumption of sweet carbohydrates by the determined genotype. The method for identification of a human who has the predisposition for increased consumption of carbohydrates comprises:

a) detection of human ADRA2A genotype from the biological sample and the determination whether there is a nucleotide C or G at the position of −1291 or a polymorphism that is in linkage disequilibrium with −1291 ADRA2A.
b) detection whether both alleles of the named gene position carry nucleotide C or there is nucleotide C in one allele and nucleotide G in the other or there is nucleotide G in both alleles. If the position of −1291 ADRA2A gene is homozygous for G allele, then the named human has a predisposition for increased consumption of carbohydrates and the predisposition to develop a metabolic, psychiatric or neurological disease or disorder, or obesity.

Given the importance of the alpha-2A adrenoreceptors in modulating a variety of physiological functions, there is a need in the art to identify polymorphisms that may stand behind the development of different diseases and to correlate the identity of these polymorphisms with signaling functions of alpha-2A adrenoreceptors. It is known to those skilled in the art that any nucleotide polymorphism in linkage disequilibrium can be used as a marker for the polymorphism behind a disease or disorder. The present invention addresses some of those above mentioned needs. The present invention is useful for determining an individual's risk for developing a disease or disorder and assist the clinician in diagnosing and prognosing diseases. This invention provides also a method for managing the dietary intake of nutritional energy of a human identified according to the above disclosed method, wherein the said human is prescribed to decreased intake of carbohydrates.

The DNA that makes up human chromosomes provides the instructions that direct the production of all proteins in the human body. These proteins carry out the vital functions of life. Variations in DNA sequences in promoter or coding region of the gene may produce various mutations, potentially affecting the normal function of cells and the whole body. Although environment often plays a significant role in the disease, variations or mutations in the DNA of an individual are directly related to almost all human. Knowledge of genetics will help unravel the genetic bases of disease and be useful in treatment. The present invention addresses some of those needs. The term “alpha 2a adrenergic receptor polymorphism” is the term of art and refers to at least one polymorphism in the nucleic acid sequence of ADRA2A gene or gene product.

There have been several reports of non-coding region polymorphisms (i.e., in the 5′ and 3′ UTR) of the human alpha-2A adrenoreceptors. In one embodiment of the present invention is the single nucleotide polymorphism at the position C-1291G. This polymorphism was originally reported at the position −1291, upstream of the origin of transcription; however, subsequent analysis has confirmed its position at −1291, upstream of the start of translation. As used herein, the allele carrying nucleic acid residue C at the position −1291 of ADRA2A is further referred as the “C” allele and the allele carrying nucleic acid residue G at the position −1291 of ADRA2A is further referred as the “G” allele.

The present investigation provides a method and a kit for identifying a human having a predisposition to develop metabolic, psychiatrical or neurological diseases or disorders, or obesity comprising detecting a human's ADRA2A genotype and assessing the human's predisposition for increased consumption of sweet carbohydrates due to the determined genotype.

The present invention refers to the polymorphism in the promoter region of the ADRA2A gene at the position −1291. This altered sequence and the initial sequence may co-exist in a species' population. The frequency of this polymorphism is different in human races. For example, the frequency of the GG genotype of C-1291G polymorphism is about 4-5 percent in Caucasians. In Afro-Africans the frequency of GG genotype is about 45-50 percent. In some instances, those changes confer neither advantage nor disadvantage to the species and multiple alleles of the sequence may be in stable equilibrium. In some instances, however, these sequence changes will confer a survival or evolutionary advantage to the species, and accordingly, the altered allele may eventually (i.e. over evolutionary time) be incorporated into the genome of many or most members of that species. As used herein, the term “polymorphism” refers to the condition in which there is a variation in the DNA sequence between some nucleotides. Typically, the polymorphism is termed as “mutation” if a variation is uncommon (less than 1%), that are rare allelic variants and may be the single basis of an inherited disease, and that may result in a gene that encodes a non-functioning protein or a protein with an altered or reduced function. Such mutations or polymorphisms include, but are not limited to, SNPs, one or more base deletions, and one or more base insertions. On the other hand polymorphisms may have no effect, have effects that are clinically silent but can be delineated with physical testing, or can alter the response to therapy. Within coding region of genes, polymorphisms may encode different amino acids or because of redundancy in the genetic code, may have no effect on the encoded amino acid. Polymorphisms occur in 5′ untranslated region (UTR), promoter region, 3′UTR, and introns as well, and in general, these are more common than coding polymorphisms. There may also exist other polymorphisms in linkage disequilibrium related to the same phenotype. In this invention, though, no pharmacologic studies were carried out to determine if this polymorphism alters the receptor function. However, the polymorphism in the promoter region of the gene may alter receptor density or expression.

In another embodiment of the present invention includes a method to determine the single nucleotide polymorphism at the promoter region of −1291 ADRA2A gene where a C allele is substituted with G allele. This nucleotide substitution causes the change in a human's eating behaviour, by consuming more carbohydrates. Specifically, subjects with GG genotype are prone to consume remarkably more sweet carbohydrates like, chocolate, nougat, caramel, ice cream, sweet cottage cheese etc. The mentioned polymorphism causes also alterations in glucose metabolism. Specifically the GG genotype effect is known to lower the blood glucose levels in human, its effect is rather small and not to blood glucose levels that can be classified as hypoglucemia. Therefore, the increased consumption of sweet carbohydrates is not directly the cause of the altered glucose levels. The direct mechanism how the C-1291G polymorphism influences eating behaviour is not yet known in the art. As adrenoreceptors are localized in the brain, pancreas, adipose tissue etc., the influence may be present at one or several previously described levels.

In another embodiment, the present invention includes a method to determine the predisposion of the human to develop metabolic, psychiatric or neurological disease or disorders or obesity. The human harbouring the GG genotype tend to consume more calories than CC or CG genotype, indicating excessive energy intake. Excessive energy intake is one of the main determinants of increased risk to different metabolic diseases like obesity, cardiovascular diseases, hypertension, type II diabetes. Increased carbohydrate intake may indicate also neurological or psychiatric disease or disorder.

In another embodiment, the present invention also provides a method for managing a human's dietary energy intake, comprising detection of a human's ADRA2A genotype, assessing the human's predisposition to develop metabolic, psychiatric or neurological diseases or disorders, or obesity and regulating (decreasing) the intake of sweet carbohydrates from the diet of the human predisposed to develop the above mentioned diseases or disorders.

The current invention can be used for diagnosis of atypical depression, more precisely the depression with craving for sweet food products such as chocolate, nougat, caramel etc. or known also as sweet carbohydrates in general.

The invention can be also used for diagnosis, prognosis, and treating diseases such as eating disorders, that could lead to unexpected weight gain, obesity, and emotional disorders that are associated with the ADRA2A gene.

Diseases Associated with ADRA2A

Alpha-adrenergic receptors play an important role in regulating a variety of physiological functions because of their distribution in many organs of the body and the brain. Thus, dysfunctional, alpha-2A receptors can predispose to, or modify a number of diseases or alter response to therapy. The present invention stems in part from the recognition that certain polymorphism(s) in the alpha-2A adrenoreceptor result in receptor molecules that could alter receptor expression or receptor density (see examples above). This altered receptor functions put an individual at risk for developing different diseases associated with the alpha 2A adrenoreceptors.

As used herein, “disease” includes but is not limited to any condition manifested as one or more physical and/or psychological symptoms for which treatment is desirable, and includes previously and newly identified diseases and other health disorders. Such diseases include, but are not limited to cardiovascular diseases such as hypertension, hypotension, arrhythmias, myocardial infarction, metabolic rate and combinations thereof, eating behaviours, drug induced or unexpected body weight gain. Central nervous system diseases are also contemplated by the present invention. Some examples of central nervous system diseases include atypical depression, hyperreactivity, anxiety, manic-depression and combinations thereof. Since alpha-2A adrenoreceptors control certain central nervous system and peripheral functions as discussed above, dysfunctional polymorphisms are likely to be important in as of yet unclassified disorders of memory and behavior.

In this invention, “diagnosis” includes determining the nature and cause of the disease, based on different signs and symptoms of the disease and laboratory findings. Such laboratory finding is the identification of (at least one) polymorphism in nucleic sequence of the ADRA2A gene. Prognosis of a disease includes determining the probable clinical course and outcome of the disease. Increased risk for the disease includes an individual's propensity or probability for developing the disease.

As used herein, “atypical depression” is a subtype major depression characterized by mood re activity—being able to experience improved mood in response to positive events. Additionally, atypical depression is characterized by reversed vegetative symptoms, namely over-eating and over-sleeping. The DSM-IV-TR, a widely used manual for diagnosing mental disorders, defines Major Depressive Disorder with Atypical Features as subtype of depression characterized by: A. Mood reactivity i.e., mood brightens in response to actual or potential positive events). B. At least two of the following: 1 Significant weight gain or increase in appetite; 2. Hypersomnia (sleeping too much, as opposed to the insomnia present in melancholic depression; 3. Leaden paralysis (i.e., heavy, leaden feeling in arms or legs); 4 Long-standing pattern of interpersonal rejection sensitivity (not limited to episodes of mood disturbance) that results in significant social or occupational impairment.

In general atypical depression tends to cause greater functional impairment than other forms depression. Atypical depression tends to occur earlier in life than other forms of depression—usually beginning in teenage years. Similarly, patients with atypical depression are more likely to suffer from other mental illnesses such as social phobia, avoidant personality disorder, or body dysmorphic disorder. Atypical depression is more common females—nearly 70% of the atypical population women.

The term “diet” refers to the sum of food consumed by a human. “Dietary habits” are the habitual decisions a human makes when choosing what foods to eat. Individual dietary habits may be more or less healthy and they play a significant role in health and mortality. A balanced diet includes several types of nutritional compounds, including carbohydrates. However, a disproportionate level of carbohydrates may contribute to obesity. Several types of low- and no-carbohydrate foods and diets may be used.

“Carbohydrates” is the term known to those skilled in the art and is used here as the nutritional compounds that are present in a wide array of food stuffs. They are a group of numerous different compounds that occur in a variety of sizes and configurations. All carbohydrates are composed of one or more sugar units linked together through glycosidic bonds. Complex carbohydrates, such as strach, are relatively large molecules consisting of numerous repeating units formed into a multi-branched chain structure. Monosaccharides and disaccharides, on the other hand, are simple carbohydrates that comprise single and double sugar units.

The term “sweet carbohydrates” refers to simple carbohydrates that comprise single and double sugar units and they taste sweet in contrast to complex carbohydrates. For example potatoes are pleasant for taste buds, and contain plenty of carbohydrates, but are not sweet.

As used herein, the term “SNP” or “single nucleotide polymorphism” refers to a genetic variation between individuals; e.g., a single nitrogenous base position in the DNA of organisms that is variable. As used herein, “SNPs” is the plural of SNP. Essentially, when one refers to DNA herein such reference may include derivatives of DNA such as amplicons, RNA transcripts, etc.

In another embodiment, the present invention includes methods of determining the risk an individual has for developing a disease. Alternatively, the present invention can be used to diagnose an individual or give prognosis to an individual with a disease. The GG genotype may predispose subjects susceptile with body weight gain in general. Alpha 2A adrenoreceptors are highly expressed in the adipose tissue, specifically in the subcutaneous adipose tissue. Lipids are mobilized from fat cells via lipolysis through the hydrolysis of triglycerides to glycerol and free fatty acids. The process is controlled by several hormones, along which catecholamines (epinephrine and norepinephrine) are the most important lipolytic ones in humans. Acting via G-protein coupled adrenergic receptors and have dual effect on lipolysis by stimulating via beta adrenoreceptors and inhibiting via alpha-2A adrenoreceptors.

In another embodiment of the present investigation, the subjects with GG genotype at the site −1291 of the promoter region of the ADRA2A gene show a different body fat mass distribution. An alpha 2A antilipolytic effect partly explains those differences between the gynoid and android pattern of fat distribution observed on average in women and men, respectively. Therefore, the proven genotype effect on increased consumption of sweet carbohydrates, weight gain, and fat distribution is probably the prerequisite to the development of the metabolic diseases (metabolic syndrome).

“Metabolic diseases” (metabolic syndrome) are defined herein as conditions, which result from an abnormality in any of the following forms: obesity, insulin resistance, hypertension, dyslipidemia, type II diabetes, and other metabolic abnormalities associated with an increased risk of atherolosclerotic cardiovascular disease in adults.

“Metabolic diseases” are often caused by single defects in particular biochemical pathways, defects that are due to the deficient activity of individual enzymes or molecular receptors leading to the regulation of such enzymes. Hence, in broader sense disturbances of the underlying genes, their products and their regulation lie well within the scope of this definition of a metabolic disease. When endocrine glands malfunction, hormone levels in the blood can become abnormally high or low, disrupting body functions. Many disorders of the metabolic syndrome are caused by malfunction of the endocrine system or hormones. Examples of such disorders are presented in the following.

The insulin-secreting cells of the pancreas respond to glucose and fatty acids. Diabetes mellitus is a disorder in which blood levels of glucose are abnormally high because the body doesn't release or use insulin adequately.

People with type I diabetes mellitus (insulin-dependent diabetes) produce little or no insulin at all. In type I diabetes more than 90 percent of the insulin-producing cells (beta cells) of the pancreas are permanently destroyed. The resulting insulin deficiency is severe, and to survive, a person with type I diabetes must regularly inject insulin.

In type II diabetes mellitus (non-insulin-dependent diabetes) the body develops resistance to insulin effects, resulting in a relative insulin deficiency.

### DESCRIPTION OF THE EMBODIMENTS

### Preparation of a Biological Sample

Nucleic acid molecules may be prepared for analysis from unpurified tissue (blood, saliva, buccal cells) using any technique known to those skilled in the art. Preferably such techniques result in the production of a nucleic acid molecule sufficiently pure to determine the presence or absence of one or more SNPs at one or more locations in the nucleic acid molecule.

When the nucleic acid of interest is present in a cell, it may be necessary to first prepare an extract of the cell and then perform further steps i.e., differential precipitation, column chromatography, extraction with organic solvents and the like in order to obtain a sufficiently pure preparation of nucleic acid. Extracts may be prepared using standard techniques in the art, for example, by chemical or mechanical lysis of the cell. Extracts then may be further treated, for example, by filtration and/or centrifugation and/or with chaotropic salts such as guanidinium isothiocyanate or urea or with organic solvents such as phenol and/or HCCl.sub.3 to denature any contaminating or potentially interfering proteins. When chaotropic salts are used, it is usually desirable to remove the salts from the nucleic acid-containing sample. This can be accomplished using standard techniques in the art such as precipitation, filtration, size exclusion chromatography and the like.

In the present embodiment, denaturation and renaturation (annealing) of the sample DNA was performed. Denaturation is a process whereby a DNA molecule is converted from a two-stranded helical structure to a flexible, single-stranded structure, usually by applying heat. Renaturation is the reverse of the process, that is, cooling or otherwise reversing the process so that the single strands of DNA anneal to one another in a sequence specific manner to form double-stranded DNA.

In some instances, it may be desirable to extract and separate messenger RNA from cells. Techniques and material for this purpose are known to those skilled in the art and may involve the use of oligo dT attached to a solid support such as a bead or plastic surface. Suitable conditions and materials are known to those skilled in the art. It may be desirable to reverse transcribe the mRNA into cDNA using, for example, a reverse transcriptase enzyme. Suitable enzymes are commercially available from, for example, Invitrogen of Carlsbad, Calif., or Fermentas, Lithuania. cDNA prepared from mRNA may then be amplified.

It may be desirable to amplify one or more nucleic acids of interest before determining the presence or absence of one or more variations in the nucleic acid, though amplification is not a necessary step for genotyping utilizing the SNP genotyping methods of the present invention. Nucleic acid amplification increases the number of copies of the nucleic acid sequence of interest. Any amplification technique known to those of skill in the art may be used in conjunction with the present invention including, but not limited to, polymerase chain reaction (PCR) techniques. PCR may be carried out using materials and methods known to those of skill in the art.

The nucleic acids used in the methods of the invention may be labeled to facilitate detection in subsequent steps. Labeling may be carried out during an amplification reaction by incorporating one or more labeled primers into the amplified sequence. The nucleic acids may be labeled following amplification, for example, by covalent attachment of one or more detectable groups. Any detectable group known to those skilled in the art may be used, for example, fluorescent groups, ligands and/or radioactive groups. An example of a suitable labeling technique is to incorporate nucleotides containing labels into the nucleic acid of interest using a terminal deoxynucleotidyl transferase (TdT) enzyme. For example, a nucleotide—preferably a dideoxy nucleotide—containing a label is incubated with the nucleic acid to be labeled and a sufficient amount of TdT to incorporate the nucleotide. A preferred nucleotide is a dideoxynucleotide—i.e., ddATP, ddGTP, ddCTP, ddTTP, etc, having a biotin label attached.

The nucleic acid sequences to be analyzed by the methods of the present invention may be subjected to other treatments before labeling. For example, in some cases, it may be desirable to fragment the amplified sequence prior to hybridization with an oligonucleotide array. Fragmentation of the nucleic acids generally may be carried out by physical, chemical or enzymatic methods that are known in the art. Suitable techniques include, but are not limited to, subjecting the amplified nucleic acids to shear forces by forcing the nucleic acid-containing fluid sample through a narrow aperture or digesting the nucleic acid with a nuclease enzyme. One example of a suitable nuclease enzyme is Dnase I. After amplification, the nucleic acid may be incubated in the presence of a nuclease for a period of time designed to produce appropriately sized fragments. The sizes of the fragments may be varied as desired, for example, by increasing the amount of nuclease or duration of incubation to produce smaller fragments or by decreasing the amount of nuclease or period of incubation to produce larger fragments. Adjusting the digestion conditions to produce fragments of the desired size is within the capabilities of a person of ordinary skill in the art. The fragments thus produced may be labeled as described above.

### Methods for Detecting SNP

There are several methods for SNP genotyping known in the art. For example, DNA sequencing is well known and generally available in the art and may be used to determine the location of SNPs in a genome. Those methods include, but are not limited to capillary electrophoresis systems, hybridization techniques, invader technology, rolling circle amplification, use of the 5″-exonuclease activity of a DNA polymerase, solid phase amplification and microsequencing of the amplification product, single strand conformation polymorphisms, allele specific oligonucleotide ligation, and allele specific hybridization. For detailed description of the analysis techinques see U.S. Pat. No. 7,189,512 (Borat N, Porat B), or non PCR used methods that are for example, but not limited to invasive cleavage of oligonucleotide probes.

### EXAMPLE

Identification of a human with the predisposition to develop metabolic, psychiatric or neurological disease or disorder or obesity.

### 1. Screening for the −1291 Polymorphism at Human ADRA2A Gene.

Materials and methods of genotype detection For examination, DNA from 1171 randomly selected sample of 9 and 15 year old children participating in the European Heart Study, was utilized.

A nucleotide substitution at the promoter region of ADRA2A was identified as known to those skilled in the art. The MspI restriction enzyme identifies C nucleotide substitution to G at the position −1291 of the ADRA2A gene. The PCR mixture contained about 100 ng DNA, (MgCl₂, 0.1 mMdNTTP, PCR buffer [75 mM Tris HCl pH 8.8; 20 mM (NH)₂SO₄; 0.01% Triton X100; 0.5% Ficoll 400; 1 mM Tartrazine]; 0.2 mM primers each; 0.5 U Taq Polymerase (Fermentas, Lithuania)). PCR amplification procedures are the following: the initial denaturation step at 94° C. for 3 minutes, followed by 35 cycles of denaturation at 94° C. for 30 s, annealing at 60° C. for 45 s, and extension at 72° C. for 45 s.

The used forward primer 5″-TCA CAC CGG AGG TTA CTT CCC TCG-3″ and the reverse primer 5″-TCC GAC GAC AGC GCG AGT-3″ generated a PCR product of 552 bp. After each PCR amplification, the PCR product was digested overnight at 37° C. after adding 6 U of the restriction enzyme MspI to the PCR mixture that would cut the product into five fragments (5, 62, 116, 165, and 174 bp). The 174 by fragment was cut into two bands in the presence of the G-1291 allele. Resulting fragments were separated by electrophoresis in 3% agarose gels. Each gel stained with ethidium bromide was run for 30 minutes at 180V, and photographed under UV transmitted light. The allele without the MspI restriction site was designated as C-allele and G-allele is with the restriction of the MspI site.

The kit of the invention comprised PCR buffer, primers, nucleotides, Taq polymerase and MspI restriction endonuclease (i.e. means for detecting the genetical polymorphism at the position −1291 of ADRA2A gene).

### 2. Assessment of Human Predisposition to Increased Consumption of Carbohydrates.

The dietary habits of a human can be determined used any known method known in the art. In the present embodiment, the questionnaire of the consumed food and food supplements of 24 hours was used with the indications to the type and the amount of food. Next day a face-to-face interactive interview was performed. The size of the portion that was not indicated on the food record was estimated using pictures of portion sizes. Nutrient intake was then analysed with a software Micro-Nutrica 2.0.

The intake of sweet food products (e.g. chocolate, candies, nougat) and sour milk products was higher in GG genotype. No other food products were consumed differently among three genotypes. Daily caloric intake was not significantly different, but a tendency for higher intake was found in the GG group. No difference was observed in body composition or physical activity level among the genotypes.

Allele and genotype frequencies 1171 Caucasian subjects are presented in Table 1.

TABLE 1
Allelic and genotype frequencies of
the 1171 Caucasian boys and girls

| | |
|---|---|
| Alleles | Genotypes |

| | n | C | G | CC | CG | GG |
|---|---|---|---|---|---|---|
| Boys | 536 | 0.79 | 0.21 | 0.61 | 0.34 | 0.05 |
| Girls | 635 | 0.77 | 0.23 | 0.57 | 0.39 | 0.04 |

The results indicate that homozygous −1291G genotype had a significant effect on the consumption of sweet food products, of the type for which the subjects may show their own preference (Table 2).

TABLE 2
Daily dietary intake of sweet food products
according to ADRA2A-1291 genotype.

| | ADRA2A genotype | | | |
|---|---|---|---|---|
| | CC | CG | GG | |
| | n = 694 | n = 429 | n = 48 | P |
| Caloric intake (Kcal) | 2134.3 ± 889.3 | 2062.8 ± 862.5 | 2256.7 ± 1051.4 | 0.059 |
| Sugar added to | 31.1 ± 26.9 | 31.7 ± 36.1 | 34.5 ± 42.00 | 0.761 |
| food (g) | | | | |
| Sweet food | 21.8 ± 43.5 | 21.6 ± 32.2 | 39.1 ± 82.5^{a} | 0.015 |
| products* (g) | | | | |
| Sour milk | 58.1 ± 113.7 | 48.7 ± 95.4 | 88.4 ± 159.7^{a} | 0.048 |
| products (g)** | | | | |

Values are expressed as mean ± SD.
^{a}significantly different from CC and CG genotypes, p < 0.05;
*sweet food products comprise sweet carbohydrates,
**60% of the consumption of sour milk products is the consumption of sweet yoghurt and sweet cottage cheese.

### Management of Nutritional Energy Intake

### 1. Screening for the C-1291G Human ADRA2A Genotype Genotyping was Carried Out According to Example 1.

### 2. Assessment of Human's Predisposition to Develop Metabolic, Psychiatric or Neurological Diseases or Disorders or Obesity.

Consumption of sugar added to food prepared at home was not different between genotypes, but this indeed depends on the dietary habits of the family. Moreover, as the sour milk products consumed were known to be to a large extent rather sweet, we examined the effect of genotype and a similar effect was found. Subjects with the GG genotype have been found to be more susceptible to weight gain under clozapine treatment. In contrast, our study did not reveal any differences in body compositional parameters among different genotypes in a population-representative sample of children. However, higher consumption of sweet food products of GG carriers in childhood may result in different body composition in adulthood. Moreover, harbourers of the GG genotype, indeed, tended to consume more calories (Table 2), which puts them to a clear risk to develop obesity. C-1291G polymorphism had marginal effects on body composition in white adult subjects. However, only the data of the carriers or non carriers of the G allele were compared, probably because the GG genotype was relatively rare. The GG genotype is more common in black people, and had a clear influence on body composition.

3. Prescription of Decreased Intake of Carbohydrates from the Diet

A balanced diet includes several types of nutritional compounds, including carbohydrates. However, excessive carbohydrates in the organism are converted to lipids and stored in the human body as energy reserve. Excessive carbohydrate intake may contribute to obesity and then further to cardiovascular diseases, insulin resistance, hypertension and metabolic syndrome. Increased consumption of sweet carbohydrates is one predisposer of hyperactivity and atypical depression, by means of eating disorders. Knowing the individual genotype that reveals the human's dietary predispositions helps the physicians or any other skilled person to propose the diet, where the carbohydrates are at the lesser amount as low carbohydrate diet or even non carbohydrate diet if necessary. The dietary regulation includes relaxing or suspending the diet, for example, but is not limited to a) the decreased intake of sweet carbohydrates and therefore the decreased amount of consumed calories or b) decreased amount of sweet carbohydrates, but increasing the amount of other food components that results in no change of consumed calories.

Metabolic diseases or disorders mentioned in the current invention comprise but are not limited to obesity, insulin resistance, hypertension, dyslipidemia, type II diabetes, atherosclerotic or other type of cardiovascular disease and other metabolic abnormalities.

Psychiatric or neurological diseases or disorders mentioned in this invention comprise but are not limited to atypical depression, hyperreactivity, anxiety, manic-depression and combinations thereof.

1. A method for identifying a human who has a predisposition for increased consumption of carbohydrates comprising the steps of:
obtaining a biological sample from said human;
determining a genotype of human ADRA2A gene, wherein said determining includes assessing whether there is a nucleotide C or G at position of −1291 in each allele of said ADRA2A gene, or whether there is a polymorphism that is in linkage disequilibrium with regards to position −1291 of each allele of said ADRA2A gene;
evaluating whether the position of −1291 ADRA2A gene is homozygous for G allele; and
determining whether said human has a predisposition for increased consumption of carbohydrates and the predisposition to develop a metabolic, psychiatric or neurological disease or disorder, or obesity by using information about homozygous GG genotype of said ADRA2A gene at position of −1291 of said ADRA2A gene or a homozygous genotype in linkage disequilibrium with regards to G allele at position −1291 of ADRA2A gene.

2. The method according to claim 1, wherein the carbohydrates comprise sweet carbohydrates.

3. The method according to claim 1, wherein the said metabolic diseases or disorders comprise obesity, insulin resistance, hypertension, dyslipidemia, type II diabetes, and other metabolic abnormalities associated with an increased risk to develop an atherosclerotic cardiovascular disease.

4. The method of claim 1, wherein the psychiatric or neurological diseases or disorders comprise atypical depression, hyperreactivity, anxiety, manic-depression and combinations thereof.

5. A method for managing the dietary intake of nutritional energy of a human comprising the steps of:
obtaining a biological sample from said human;
determining a genotype of human ADRA2A gene, wherein said determining includes assessing whether there is a nucleotide C or G at position of −1291 in each allele of said ADRA2A gene, or whether there is a polymorphism that is in linkage disequilibrium with regards to position −1291 of each allele of said ADRA2A gene;
evaluating whether the position of −1291 ADRA2A gene is homozygous for G allele;
determining whether said human has a predisposition for increased consumption of carbohydrates and the predisposition to develop a metabolic, psychiatric or neurological disease or disorder, or obesity by using information about homozygous GG genotype of said ADRA2A gene at position of −1291 of said ADRA2A gene or a homozygous genotype in linkage disequilibrium with regards to G allele at position −1291 of ADRA2A gene; and
if said human has said predisposition for increased consumption of carbohydrates, prescribing to said human a decreased intake of carbohydrates.

6. A kit for identification of a human with the predisposition for increased consumption of carbohydrates wherein said kit comprises a means for detecting a genetic polymorphism in the ADRA2A gene at position −1291 or a means for detecting a polymorphism that is in linkage disequilibrium with polymorphic alleles at position −1291 of the ADRA2A gene.
